(19) United States
(12) Patent Application Publication
(10) Pub. No.: US 2024/0415987 A1
(43) Pub. Date: Dec. 19, 2024

(54) METHOD FOR EXTRACTING A FLUORINATED GAS

(71) Applicant: Bracco Suisse SA, Cadempino (CH)

(72) Inventors: Carlo Felice VISCARDI, Milan (IT); Ilaria MANCINI, Milan (IT)

(21) Appl. No.: 18/719,175

(22) PCT Filed: Dec. 16, 2022
(86) PCT No.: PCT/EP2022/086497

§ 371 Date: Jun. 12, 2024

(30) Foreign Application Priority Data
Dec. 16, 2021 (EP) 21215193.0

(51) Int. Cl. A61K 49/22; F26B 5/06; F26B 21/14

(52) U.S. Cl. CPC A61K 49/223; F26B 5/06; F26B 21/14

(57) ABSTRACT

A method for extracting a fluorinated gas from a freeze-drying chamber by insufflating a second gas into said chamber, extracting the gas from said container and controlling said extraction of gas to avoid a backflow of the extracted gas into the chamber.

### TECHNICAL FIELD

The invention relates to a method and system for extracting a gas comprising a fluorinated gas from a freeze-drying chamber.

### BACKGROUND OF THE INVENTION

Gas-filled microvesicles represent a class of contrast agents particularly useful for Contrast Enhanced UltraSound imaging (“CEUS” imaging), typically in the form of suspensions of gas bubbles of nano- and/or micro-metric size dispersed in an aqueous medium. The gas is typically entrapped or encapsulated in a film-layer comprising, for instance, emulsifiers, oils, thickeners or sugars. These stabilized gas bubbles (dispersed in a suitable physiological solution) are generally referred to in the art with various terminologies, depending typically from the stabilizing material employed for their preparation; these terms include, for instance, “microspheres”, “microbubbles”, “microcapsules” or “microballoons”, globally referred to here as “gas-filled microvesicles” (or “GFM” in short).

GFM may be prepared by using different preparation methods. For instance, according to the disclosure of Ref. 1, a suspension or emulsion comprising the amphiphilic materials useful for forming the GFM is filled into a number of vials, which are then submitted to a freeze-drying process in a respective freeze-drying chamber. At the end of the freeze-drying process, the headspace of the vials is filled with a fluorinated gas (or with a mixture of a fluorinated gas with another gas-soluble in water, e.g. air or nitrogen). Typically, the gasification of the vials' headspace is obtained by saturating the freeze-drying chamber with the desired gas or mixture of gases. At the end of the gasification step, the vials are stoppered and the freeze-drying chamber is then opened, in order to collect the vials and seal them. The sealed vials with the freeze-dried product can then be stored for relatively long periods before use. Before use, the content of the vial is reconstituted by injecting a suitable physiologically acceptable carrier (e.g. saline, glucose solutions etc.) in the vial and gently agitating to form a suspension of GFM.

In general, upon opening of the chamber for collecting the stoppered vials, the gas contained in (and saturating) the chamber diffuses in the surrounding ambient and is typically evacuated with conventional ventilation systems. As observed by the Applicant, even if the volume of fluorinated gas (or gas mixture) contained in the freeze-drying chamber is relatively low, it would nevertheless be desirable to recover such gas for future uses instead of wasting it.

While the use and respective recovery of fluorinated gases is already performed in other technical fields, such as in the case of gas (e.g. sulfur hexafluoride) removal from high-voltage electric appliances, the Applicant has observed that the recovery of fluorinated gases in the case of GFM's manufacturing poses a number of technical issues, in particular in connection with the specific manufacturing process and the constraints due to the pharmaceutical use of the GFM.

First of all, Applicant has observed that a simple extraction by suction of the fluorinated gas (as typically performed in other technical fields) at the end of the freeze-drying process would create a negative pressure (i.e. below ambient pressure) inside the freeze-drying chamber, which may not be compatible with respect to the pressure of gas contained in the stoppered vials; as observed by the Applicant, any such negative pressure should in fact be avoided, as it may cause the pop-up of the stoppers from the vials. Also, even if the stopper in the vial could be to somehow kept pressed into the vial by mechanical means, there is the risk of gas leakage from the vial to the external ambient when this latter is subjected to a negative pressure.

Furthermore, the environment where the freeze-drying chamber is located is a sterile environment, which is in general physically isolated from other neighboring manufacturing environments. As observed by the Applicant, during the extraction of the gas from the freeze-drying chamber, a possible backflow of gas from the extraction line into the freeze-drying chamber (e.g. due to malfunctioning of the extracting device) may occur. This inconvenience is particularly undesired and should be avoided in order to maintain the desired sterility inside the freeze-drying chamber.

For at least these technical problems, there is the need to develop a new method for extracting a fluorinated gas from a from a freeze-drying chamber, which takes into account the presence of vials comprising a freeze-dried pharmaceutical product inside the chamber and more in general the specific requirements of pharmaceutical grade manufacturing.

### SUMMARY OF THE INVENTION

An aspect of the invention relates to a method for extracting a gas from an inner volume of a freeze-drying chamber, said chamber comprising a plurality of stoppered vials comprising a freeze-dried product, said stoppered vials and said chamber containing a gas at a predetermined pressure, said gas comprising a fluorinated gas, wherein said method comprises:
insufflating a volume of a second gas into said chamber at a pressure higher than the said predetermined pressure;
extracting the gas from said chamber; and
controlling said extraction of gas to avoid a backflow of the extracted gas into the chamber.

Preferably said second gas is nitrogen.

In an embodiment, the extraction of the gas is carried out by an extracting system comprising: (i) extracting means for extracting the gas from the chamber; and (ii) a first valve, positioned on an extracting line between the extracting means and the chamber, to control the flow of gas from the chamber to the extracting means. In a preferred embodiment, said first valve closes if the pressure of the extracting system exceeds a predetermined value.

In an embodiment said control of gas backflow is accomplished by maintaining the extracting system at a pressure lower than the pressure of the flow of the second gas being insufflated inside the chamber.

In a further embodiment, said extracting system comprises a second valve, positioned on an auxiliary line connected to the extracting line and located between the first valve and the extracting means, which opens if the pressure of the extracting system exceeds a predetermined value. In a preferred embodiment, said extracting system further comprises a third valve, positioned on the extracting line between said first valve and the extracting means.

In an embodiment, said extracting system further comprises compressing means for compressing the gas after extraction thereof.

The extracting means is operated to extract the gas from the chamber while maintaining a pressure in the extracting line downstream from said first valve lower than the pressure inside the chamber receiving the nitrogen gas.

In an embodiment, the volume of the second gas is insufflated into the chamber is three times the volume of the first gas contained in the chamber.

Typically, the freeze-dryer is located in a sterile environment. Preferably said first valve is also located in the sterile environment, more preferably at the boundary of said sterile environment.

Another aspect of the invention relates to a method for manufacturing a freeze-dried composition suitable for the preparation of a suspension of stabilized gas-filled microbubbles, said composition comprising (i) an amphiphilic material; and (ii) a freeze-drying protecting component, which comprises:
a. preparing a liquid mixture comprising said amphiphilic material and said freeze-drying protecting component in a solvent and filling a plurality of vials with said liquid mixture;
b. introducing said vials into a freeze-drying chamber;
c. freeze-drying the liquid mixture to remove said solvent and obtain a freeze-dried product;
d. saturating the freeze-drying device and the headspace of said vials with a gas comprising a fluorinated gas;
e. stoppering the vials;
f. extracting the gas from the chamber according to the process described above; and
g. removing the vials from the chamber.

A further aspect of the invention relates to a freeze-drying system comprising:
a. a freeze-drying chamber; and
b. an extracting system for extracting a first gas from said chamber, said extracting system comprising:
   an inlet line connected to said chamber and a respective valve for supplying a second gas to said chamber;
   an output through which said first gas is extracted from said chamber;
   a second valve connected to said output;
   an extracting line connected to said second valve; and
   extracting means connected to the opposite end of said extracting line, said extracting means comprising an output for collecting the extracted gas;
said freeze-drying system further comprising a control system for controlling the extraction of said first gas from the freeze-drying chamber and the supply of said second gas thereto.

### FIGURES

FIG. 1 illustrates a schematic example of a system suitable for the process of the invention.

FIG. 2 illustrates a schematic example of an alternative system suitable for the process of the invention.

### DETAILED DESCRIPTION OF THE INVENTION

GFM can be prepared, for instance, by reconstituting a freeze-dried product with a physiologically acceptable liquid in the presence of a fluorinated gas, under gentle agitation. The freeze-dried product, comprising the materials suitable for forming the GMF stabilizing layer (typically amphiphilic materials, such as lipids, phospholipids, fatty acids), preferably in admixture with a freeze-drying protecting component (e.g. PEG), is generally contained in a stoppered vial, in contact with a gas comprising a fluorinated gas.

The preparation of the freeze-dried product generally entails the preparation of a liquid mixture (e.g. in the form of a suspension or emulsion) containing the desired materials, such as an amphiphilic material and a freeze-drying protecting component. Suitable preparations of liquid mixtures are disclosed for instance in Ref. 1. As mentioned in Ref. 1, amphiphilic materials useful for preparing the above liquid mixtures comprise a phospholipid, while suitable freeze-drying protecting components comprise polyethylene glycol.

The liquid mixture is then distributed into a plurality of glass vials which are inserted (e.g. arranged on one or more superposed trays) into the chamber of a freeze-drying device, to undergo the freeze-drying process. The freeze-drying process typically includes a first freezing step (primary drying), where the vials are rapidly cooled at few tenths of ° C. below 0° C. (e.g. between −30° and −70° C.) at reduced pressure (e.g. between 0.1 and 0.8 mbar), and a second step, where the temperature is increased above 0° (e.g. room temperature, preferably not higher than 35° C.) while maintaining the freeze-drying chamber under vacuum.

At the end of the freeze-drying process, a fluorinated gas (or a gas mixture containing a fluorinated gas) is introduced into the (chamber of the) freeze-drying device and the chamber, as well as the headspace of the vials contained in the chamber, is saturated with such gas, at about atmospheric pressure. Depending on the type of final GFM product, the gas may be either a fluorinated gas (i.e. either a single fluorinated gas or a mixture of two or more fluorinated gases) or a mixture of a fluorinated gas with another non-fluorinated gas. Examples of suitable physiologically acceptable gases include fluorinated gases (such as SF₆, C₃F₈, C₄F₁₀ or mixtures thereof), optionally in admixture with air, nitrogen or carbon dioxide (e.g. in respective volume ratios of from 10/90 to 90/10), preferably in admixture with nitrogen.

Once the saturation of the chamber and vials with the desired gas is achieved, the gasified vials are stoppered, e.g. the vials are closed with respective rubber stoppers by means of stoppering means that are provided inside the freeze-dryer. For instance, the rubber stoppers may be initially superposed on the vials' openings and then pushed into the vials by suitable means (e.g. by raising the tray containing the vials towards a contact surface, such as the supporting element of a corresponding upper tray). The content of the stoppered vials (including the gas headspace) is thus substantially isolated from the surrounding ambient of the freeze-drying chamber.

After termination of the freeze-drying step (before or after gasification and/or stoppering of the vials) the vials may undergo an additional heating step, e.g. at a temperature higher than 35° C., for e.g. at least 8 hours, as described in Ref. 1.

As mentioned above, before opening the freeze-drying chamber for collecting the stoppered vials, the fluorinated gas has to be collected for the subsequent purification and recovery thereof.

As mentioned before, a simple extraction of the gas from the freeze-dryer would generate a negative pressure (vacuum) inside the freeze-drying chamber, with the result that the stoppers of the vials could pop-up due to the higher pressure of the gas inside the vials. Also, even if the stoppers could be forced to remain in place, the relatively high negative pressure generated in the chamber may nevertheless determine a possible leaking of the gas contained at ambient pressure inside the vials.

To avoid this, the process of the invention provides for the insufflation of a second gas (nitrogen in particular) at a pressure slightly higher than the pressure of the gas already present inside the chamber, in order to displace the gaseous content of the chamber towards the outside of the chamber. The method of the invention further provides suitable conditions for avoiding any accidental backflow of the extracted gas into the freeze-drying chamber, which may jeopardize the sterility of the chamber.

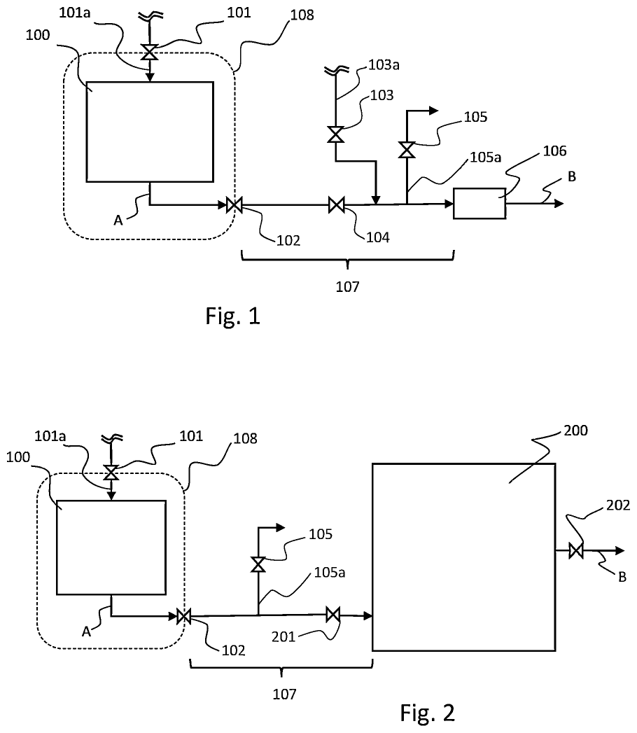

FIG. 1 illustrates a schematic example of an extraction system useful for performing the process according to the invention.

In the system illustrated in FIG. 1, an insufflating line 101a, connected to a nitrogen reservoir (not shown) and comprising valve 101 is connected to a freeze-drying chamber 100. Chamber 100 is connected (through chamber's outlet A and control valve 102) to extracting line 107, which comprises optional valve 104. An extracting device 106 is located at the end of the extracting line; the extracted gas is then directed from exit B of device 106 to a storage reservoir or to a purification/recovery system (not shown). An optional first auxiliary line 103a, comprising valve 103, is connected at one end to a nitrogen supply reservoir (not shown) and at the other end to the extracting line 107. A second auxiliary line 105a, comprising respective valve 105, is connected at the one end to the extracting line 107 and at the other end to a waste reservoir (not shown) or to the external atmosphere.

At the end of the freeze-drying and gasification process as described above, the atmosphere of the freeze-drying chamber 100 (internal volume of e.g. 1 m³ or higher, depending on the manufacturing needs), as well as the headspaces of the stoppered vials contained therein, are thus saturated with the gas comprising a fluorinated gas. The gas inside the freeze-drying chamber is typically at about ambient atmospheric pressure or slightly lower (e.g. about 0.05 less) than atmospheric pressure. The freeze-drying chamber 100 is typically located in a sterile environment 108, schematically shown in dotted line in FIG. 1.

Chamber 100 is connected (through outlet A and extraction line 107) to device 106, which comprises extracting means for extracting the gas contained within the freeze-drying chamber and sending it through exit B to a storage reservoir or to a purification/recovery system (not shown). In a preferred embodiment, device 106 preferably further comprises compressing means for compressing the gas at a pressure higher than atmospheric pressure (e.g. 10 bar, preferably 15 bar or higher, e.g. up to 20 bar) before sending it to the storage reservoir or the purification/recovery system; for instance, device 106 is a compressor, e.g. a piston compressor, typically oil-free and hermetically gas-tight. Optional cooling means (e.g. a heat exchanger), not shown, for providing an initial cooling of the gas (e.g. 20° C. or lower) before the storage or the purification/recovery process thereof may be provided within device 106 or downstream thereof as a separate device.

Device 106 may preferably be activated before the beginning of gas extraction from the chamber, e.g. by circulating nitrogen gas from optional valve 103 connected to a nitrogen reservoir (not shown) via optional first auxiliary line 103a, in order to be operative when the gas extraction from the chamber starts. In an embodiment, first auxiliary line 103a may be alternatively connected to exit B of device 106 (e.g. through a suitable switch valve, after having been initially connected to the nitrogen reservoir for the starting of the system activation) to form a closed loop for the nitrogen circulation within device 106. For instance, activation of device 106 may be initiated during the gasification process of the freeze-drying chamber. It should be noted that at this point control valve 102, which is positioned in (particularly at the boundary of) the sterile environment 108 and guarantees the sterility of the system by avoiding possible backflows of gas, is still closed. Similarly, also optional valve 104 remains closed during activation of device 106. During activation of device 106, the pressure of the extracting line downstream from optional valve 104 (or from valve 102, if valve 104 is not present) is kept at a pressure corresponding to the operative pressure applied during the extraction process. As explained in detail in the following, such operative pressure is lower than the operative pressure inside chamber 100 during the extraction process. Optional control valve 104 has substantially the same function as control valve 102 and is preferably present in order to provide an additional protection for valve 102 in case of malfunctioning of the system, by avoiding undesired backflow of gas towards the sterile zone, as well as for providing an additional control on the flow of extracted gas.

Once device 106 is ready for extracting the gas from the chamber, valve 101 (positioned on insufflating line 101a, inside or preferably at the boundary of the sterile zone) is opened and nitrogen is insufflated into the free-drying chamber through a respective inlet (preferably positioned at the top of the freeze-drying chamber), at a pressure slightly higher (e.g. 0.05 bar or higher) with respect to the pressure of the gas already present inside the chamber and inside the stoppered vials (i.e. the “predetermined pressure”); preferably, the pressure of the nitrogen entering the chamber 100 is higher than the atmospheric pressure, e.g. 0.05 bar higher than atmospheric pressure. When the pressure inside the chamber reaches the pressure of the nitrogen flow entering therein (as determined for instance by means of a pressure sensor disposed inside the chamber), the extracting system is ready for starting the gas extraction. At this point, the pressure of the system upstream of valve 102 corresponds to the pressure inside the chamber 100. While it is important to maintain the pressure inside the chamber slightly higher than the pressure of the gas into the stoppered vials during the extraction process (during the whole duration of the extraction process), for the reasons explained above, it is nevertheless also important to avoid an excessive overpressure, which may result in possible leaking of the flowing gas (e.g. nitrogen) through the stopper into the vials. Accordingly, the pressure inside the chamber during the extraction process is preferably not more than 0.2 bars higher than the pressure of the gas in the vials, preferably not more than 0.15 bars higher. Such pressure may be suitably controlled by modulating the opening of inlet valve 101. Once the desired pressure inside the chamber is reached, control valve 104 (if present) and control valve 102 are gradually opened, to allow extraction of the gas from the chamber. At the same time, if present, optional valve 103 on the optional auxiliary line 103a is gradually closed. Valve 101 remains opened during the extraction process, to provide continuous flow of nitrogen at the predetermined pressure inside chamber 100. During the extraction process, the settings of the system are controlled in order keep the pressure in the extracting line 107 at a value lower than the pressure inside the chamber and in outlet A (e.g. 0.05 bar or lower), so to avoid possible gas backflow into the chamber. For instance, when the pressure of the gas in the freeze-drying chamber is at atmospheric pressure, said pressure in the extraction line 107 (between valve 102 and device 106) is kept below ambient pressure, e.g. 0.05 bar lower than atmospheric pressure. Such control of the pressure may be performed, for instance, by suitably setting the parameters of device 106, in combination with the controlled opening of valves 102 and 104; for instance, if device 106 is a compressor, its extraction/compression parameters can be easily set as a function of the flow rate and pressure of insufflated nitrogen (in combination with the controlled opening of the valves) in order to maintain the desired pressure differential between extracting line 107 and chamber 100. Pressure sensors and flow rate meters can be positioned in the system for providing the necessary information about the status of the system, for instance on insufflating line 101a, inside chamber 100 and/or on extracting line 107.

During gas extraction from the freeze-drying chamber, the pressures and flow rates along the extraction line can be monitored in a substantially continuous manner by means of the above mentioned pressure sensors and flow meters; the whole system and process may then be controlled in a substantially automatic manner by means of an automated control system, for instance by means of a processor which receives information about flow rates and pressure values and controls the degree of opening/closing of each valve. The information may be either manually entered in the control system or received (e.g. as an electric signal) from valves or sensors of the extraction system. For instance, once the control system receives the information (e.g. manually entered by an operator) that the gasification of the freeze-dryer is completed, the control system may automatically send respective “opening” signals to valve 101, for beginning the insufflation o nitrogen. Similarly, the control system may adjust the operational parameters of device 106, and/or the opening degree of valves 101, 102 and/or 104, as a function of values of pressure and flow rate measured by the various sensors and flow meters, to keep the desired pressure differential between the extraction line and the chamber. Furthermore, if any undesired overpressure is detected in the extraction line (e.g. a differential pressure of less than 0.05 mbar with respect to the pressure inside the chamber, or a pressure above 0.95 bar), the processor intervenes and sends a signal to automatically close valve 102 (and 104) to avoid backflow of the extracted gas into the freeze-drying chamber. As mentioned above, while the presence of valve 102 would be generally sufficient for a correct working of the extracting system, optional valve 104 nevertheless provides an enhanced control on the flow of gas in the extracting line 107.

The extracting system may further comprise optional valve 105, positioned on lateral line 105a; said valve is typically a safety check valve which remains normally on the closed position, unless an undesired overpressure is detected in the extraction line (e.g. in case of malfunctioning of the compressor); if such an event occurs, valve 105 automatically opens to expel the gas from the extraction line 107, in order to remove the overpressure and avoid any possible backflow; this safety check valve may intervene either in combination with the automatic closure of valve 102 (and 104, if present) or in case such an automatic closure is accidentally delayed or not effected.

An example of normal operational pressures in the extracting line during gas extraction may be the following: (i) inside chamber 100: 1.05 bars; (ii) on the extracting line: 0.95 bars; (iii) at the exit B of device 106: 20 bars

Once the fluorinated gas has been removed from the freeze-drying chamber, the flow of nitrogen from valve 101 is stopped. However, shortly before closing valve 101, valve 102 is first closed to avoid any backflow of gas into the chamber. The end of the fluorinated gas extraction can be determined by conventional means, e.g. by measuring the residual concentration of the fluorinated gas in the flow of gas exiting the freeze-drying chamber, e.g. by an online densitometer disposed on the extracting line. Alternatively, it may be considered that the initial content of gas in the freeze-drying chamber has been substantially extracted after a volume of nitrogen of at least three times the volume of the freeze-drying chamber has been insufflated into the chamber; in the practice, the total volume of extracted gas corresponds to about three times the volume of the chamber (one volume of gas initially present in the chamber and two volumes of insufflated nitrogen gas; the third volume of nitrogen will remain in the chamber at the end of the extraction process).

The chamber 100, which will be at this point saturated only with nitrogen, can thus be opened and the vials removed from the chamber. Typically, the vials are then subsequently sealed with a metallic clamp, to firmly block the stopper.

At the exit B from device 106, the gas extracted from the freeze-drying chamber 100 (which comprises a mixture of fluorinated gas and nitrogen) can then be collected (preferably under pressure, e.g. at about 20 bars) in one or more suitable gas storage reservoir (e.g. gas cylinders,), from which it may be subsequently transferred to a purification/recovery system. Alternatively, exit B may be directly connected to a system for the purification and recovery of fluorinated gas, so to perform the whole extraction/purification/recovery process in a continuous manner.

FIG. 2 shows a schematic representation of an alternative system suitable for performing the extraction of gas from a freeze-drying chamber according to the invention. In the system illustrated in FIG. 2, the elements indicated with the numbering from 100 to 108 correspond substantially to the same respective elements described in connection with FIG. 1, with analogous functions (with the exception of auxiliary line 103a and respective valve 103, optional valve 104 and device 106, which are not included in the embodiment of FIG. 2). Differently from the system of FIG. 1, the extracting device 106 at the end of the extracting line 107 has been replaced 2 by a container 200 for receiving the gas extracted from chamber 100. The container 200 has preferably a larger inner volume than the one of chamber 100; in particular such volume is preferably larger than at least three times the volume of chamber 100, preferably about at least 3.5 times larger and even more preferably about at least four times; up to e.g. five times. The system of FIG. 2 further comprises valve 201, located on the entry line of container 200, and valve 202 located on the exit line of the container. According to this embodiment, the gas is flowing from chamber 100 to container 200 simply by differential pressure, container 200 thereby acting (in combination with valve 201) as suitable extracting means for extracting the gas from chamber 100. In particular, before beginning the extraction process, vacuum is created inside the container 200, typically from about 0.005 bar to about 0.02 bar, e.g. about 0.01 bar; typically vacuum may be created by closing valve 201 while opening valve 202 and connecting exit line B to a gas extractor (in particular a vacuum pump). As described before in connection with the embodiment of FIG. 1, the extraction process is initiated by opening valve 101 on insufflating line 101a, to insufflated nitrogen into the freeze-drying chamber; as described above, nitrogen is insufflated at a pressure slightly higher (e.g. 0.05 bar or higher) with respect to the pressure of the gas already present inside the chamber and into the stoppered vials (i.e. the “predetermined pressure”). Valves 201 and 102 are then progressively opened (while valve 202 remains closed) thus letting the gas flow by differential pressure from the freeze-drying chamber towards container 200. As the extraction process goes on, the inner volume of the container will begin to be filled with the extracted gas and the pressure inside the container will increase accordingly. As observed by the applicant, in order to provide an efficient extraction of the gas, a certain differential pressure between the chamber 100 and the container 200 shall be maintained all along the extraction process, and in particular close to the end thereof. In particular, it is preferred that at the end of the extraction process the differential pressure between chamber and container is of at least 0.1 bar, more preferably of at least 0.2 bar; for instance, when the pressure of the gas entering inside the freeze-drying chamber is about atmospheric pressure (e.g. 1.05 bar), the pressure inside container 200 at the end of the extraction process shall preferably be of 0.9 bar or less, more preferably of 0.8 bar or less. As mentioned above, the end of the extraction process can be determined by conventional means, e.g. by measuring the residual concentration of the fluorinated gas in the flow of gas exiting the freeze-drying chamber, e.g. by an online densitometer disposed on the extracting line. Alternatively, it may be considered that the initial content of gas in the freeze-drying chamber has been substantially extracted after a volume of nitrogen of at least three times the volume of the freeze-drying chamber has been insufflated into the chamber. Accordingly, as mentioned above, the volume of container 200 is preferably more than 3 times (more preferably at least 3.5 times and even more preferably at least 4 times) larger than the volume of chamber 100, in order to allow the extracted gas to fill the container while maintaining the desired minimum differential pressure between chamber 100 and container 200 at the end of the extraction.

Similarly to the embodiment described in connection with FIG. 1, also in this case the pressures and flow rates along the extraction line can be monitored in a substantially continuous manner by means of pressure sensors and flow meters and the whole system and process may be controlled in a substantially automatic manner by means of an automated control system. In particular the opening of valves 101, 102 and 201 may be controlled for suitably modulating the flow of gas into and from the freeze-drying chamber 100, in order to maintain the desired pressure gradient along the extraction line during the extraction process. Alternatively, a flow orifice may be added on the extracting line for setting a predetermined gas flow and pressure gradient, thus avoiding the need of modulating the valves opening.

Once the extraction process is completed, valves 201, 102 and 101 are closed.

The extracted gas contained in container 200 can then be collected in suitable storage containers (e.g. gas cylinders) for the subsequent treatments, as mentioned above. Exit B may thus be connected to a suitable gas extractor, e.g. a vacuum pump (by opening valve 202), for extracting the gas from container 200. In this way, (as valve 201 remained closed) container 200 is subjected to progressive vacuum; when the vacuum reaches the desired value, valve 202 is closed and container 200 is ready for the next extraction cycle. The extracted gas may then be sent to a respective (one or more) gas cylinder, preferably under pressure (e.g. about 20 bars) by using a compressor. Alternatively, the gas extracted from container 200, as described above, may be directly sent by means of a compressor to a system for the purification and recovery of fluorinated gas. In certain embodiments, the gas extractor and gas compressor may be a single device performing both extraction and compression of the gas.

Any known method and system suitable for separating fluorinated gases from other gases (in particular from nitrogen), purifying the gas and recovering it can be used. A review (with respective reference document) of the various separation, purification and recovery methods is provided for instance under chapter 2 of Ref. 2. Suitable methods include for instance cryogenic distillation or cryogenic freezing.

If desired, the outlet line A of the freeze-drying chamber may also be used during the freeze-drying process, e.g. for extracting air and creating the desired vacuum inside the freeze-drying chamber; in this case, outlet A may be modified by e.g. by connecting an additional line thereto (not shown in FIG. 1), with respective control valves and extracting means for vacuum. Similarly, the insufflating line 101a may be analogously modified and used for introducing the fluorinated gas at the end of the freeze-drying process. Alternatively, independent extracting and/or insufflating lines (not shown in FIG. 1) connected to the freeze-drying chamber may be used for providing respective vacuum and insufflation of fluorinated gas during the manufacturing process.

### CITED REFS

Ref. 1: WO2020/229642
Ref. 2: C. Y. Chuah et al., “Potential of adsorbents and membranes for SF6 captures and recovery: a review”, Chem. Eng. Journal, 404 (2021) 126577.

1. A method for manufacturing a freeze-dried composition suitable for the preparation of a suspension of stabilized gas-filled microbubbles, said composition comprising (i) an amphiphilic material and (ii) a freeze-drying protecting component, which comprises
a. preparing a liquid mixture comprising said amphiphilic material and said freeze-drying protecting component in a solvent and filling a plurality of vials with said liquid mixture;
b. introducing said vials into a freeze-drying chamber, said chamber having a respective an inner volume;
c. freeze-drying the liquid mixture to remove said solvent and obtain a freeze-dried product;
d. saturating the inner volume of the freeze-drying chamber and the headspace of said vials with a gas, comprising a fluorinated gas, at a predetermined pressure;
e. stoppering the vials;
f. extracting the gas from the chamber; and
g. removing the vials from the chamber;
wherein said step f. comprises:
insufflating a volume of a second gas into said chamber at a pressure higher than said predetermined pressure;
extracting the gas from said chamber; and
controlling said extraction of gas to avoid a backflow of the extracted gas into the chamber.

2. A method according to claim 1 wherein said second gas is nitrogen.

3. A method according to claim 1 wherein said freeze-drying chamber is located in a sterile environment.

4. A method according to claim 1, wherein the extraction of the gas is carried out by an extracting system comprising: (i) extracting means for extracting the gas from the chamber; and (ii) a first valve, positioned on an extracting line between the extracting means and the chamber, to control the flow of gas from the chamber to the extracting means.

5. A method according to claim 4, wherein said first valve closes if the pressure of the extracting system exceeds a predetermined value.

6. A method according to claim 4 wherein said first valve is located at a boundary of said sterile environment.

7. A method according to claim 4, wherein said control of gas backflow is effected by maintaining the extracting system at a pressure lower than the pressure of the second gas being insufflated inside the chamber.

8. A method according to claim 5 wherein said extracting system further comprises a second valve, positioned on an auxiliary line connected to the extracting line in a location between the first valve and the extracting means, which opens if the pressure of the extracting system exceeds a predetermined value.

9. A method according to claim 4 wherein the extracting system is operated to extract the gas from the chamber while maintaining a pressure in the extracting line downstream from said first valve lower than the pressure inside the chamber receiving the second gas.

10. A method according to claim 4 wherein said extracting system further comprises compressing means for compressing the gas after extraction thereof.

11. A method according to claim 1 wherein the volume of the second gas insufflated into the chamber is three times the inner volume of the freeze-drying chamber.

12. A method for extracting a gas from an inner volume of a freeze-drying chamber, said chamber comprising a plurality of stoppered vials comprising a freeze-dried product, said stoppered vials and said chamber containing a gas at a predetermined pressure, said gas comprising a fluorinated gas; wherein said method comprises:
insufflating a volume of a second gas into said chamber at a pressure higher than said predetermined pressure;
extracting the gas from said chamber, wherein the extraction of the gas is carried out by an extracting system comprising: (i) extracting means for extracting the gas from the chamber; and (ii) a first valve, positioned on an extracting line between the extracting means and the chamber, to control the flow of gas from the chamber to the extracting means; and
controlling said extraction of gas to avoid a backflow of the extracted gas into the chamber.

13. A method according to claim 12 wherein said second gas is nitrogen.

14. A method according to claim 12 wherein said freeze-drying chamber is located in a sterile environment.

15. (canceled)

16. A method according to claim 12, wherein said first valve closes if the pressure of the extracting system exceeds a predetermined value.

17. A method according to claim 12 wherein said first valve is located at a boundary of said sterile environment.

18. A method according to claim 12, wherein said control of gas backflow is effected by maintaining the extracting system at a pressure lower than the pressure of the second gas being insufflated inside the chamber.

19. A method according to claim 16 wherein said extracting system further comprises a second valve, positioned on an auxiliary line connected to the extracting line in a location between the first valve and the extracting means, which opens if the pressure of the extracting system exceeds a predetermined value.

20. A method according to claim 12 wherein the extracting system is operated to extract the gas from the chamber while maintaining a pressure in the extracting line downstream from said first valve lower than the pressure inside the chamber receiving the second gas.

21. A method according to claim 12 wherein said extracting system further comprises compressing means for compressing the gas after extraction thereof.

22. A method according to claim 12 wherein the volume of the second gas insufflated into the chamber is three times the inner volume of the freeze-drying chamber.
